(19) United States
(12) Patent Application Publication
(10) Pub. No.: US 2011/0319970 A1
(43) Pub. Date: Dec. 29, 2011

(54) SOLAR TANNING SYSTEM

(76) Inventor: John P. Elliott, Bay Village, OH (US)

(21) Appl. No.: 13/171,970

(22) Filed: Jun. 29, 2011

Related U.S. Application Data
(60) Provisional application No. 61/359,533, filed on Jun. 29, 2010.

(51) Int. Cl. A61N 5/06

(52) U.S. Cl. 607/95

(57) ABSTRACT

The invention relates generally to a system for use by individuals to increase their exposure to sunlight during cool or colder weather. More specifically, the invention provides a solar tanning system including a sturdy enclosure that permits sunlight to enter the enclosure, taking advantage of the same to heat the interior of the enclosure while providing a barrier against cold outside temperatures and wind.

### BACKGROUND OF THE DISCLOSURE

This application claims the benefit of priority to U.S. Provisional Patent Application No. 61/359,533, filed Jun. 29, 2010, the disclosure of which is incorporated herein by reference in its entirety.

The invention relates generally to a system for use by individuals to increase their exposure to sunlight during colder weather. More specifically, the invention provides a solar tanning system including a sturdy enclosure that permits sunlight to enter the enclosure, taking advantage of the same to heat the interior of the enclosure while providing a barrier against cold outside temperatures and wind.

It has been known to use tanning beds in order to enhance the healthy, tan look of one's skin. Generally, such beds include fluorescent or other type lamps that emit radiation intended to cause the pigment of human skin to darken, or tan. Such beds are generally known for indoor use, and predominantly during the cold winter months. While this type of bed is available for personal, in home use, most people go to establishments, tanning salons, in order to enjoy the tanning experience. This can be expensive and inconvenient.

Alternatives have been known, including enclosures of the type disclosed in U.S. Pat. No. 6,585,751 providing a collapsible frame enclosed by a porous material that permits UV radiation to pass through. Similarly, U.S. Pat. No. 6,939,366 provides an air conditioned tanning and sauna assembly. Many available products include clear domes and reflective materials.

What is currently lacking in the area of outdoor tanning devices is an enclosure that has rigid walls, constructed of dark-colored, non-reflective material, suitable to block wind and cold air from entering the interior of the enclosure and to retain heat from natural sunlight, and to provide privacy. These and other objectives of the invention will be described in detail in the disclosure which follows.

### SUMMARY OF THE DISCLOSURE

Provided herein is a solar tanning system that is a free standing multi-lateral structure, having walls at least 2 feet in height and defining an interior chamber, wherein at least a portion of the walls is sunlight transmissive and the remaining wall portion comprises a dark, non-reflective material and the chamber has an interior temperature that is higher than the outside temperature.

In one embodiment, the solar tanning system includes rigid side and floor portions that are connected to construct an enclosure for personal use when tanning outside during cool or cold weather. In another embodiment, the structure does not include a floor, but does include sides and optionally a cover. Further, the enclosure includes frame or connector members that receive and retain the rigid side and bottom portions, such that when fully constructed the enclosure defines a rectangular chamber measuring, for example, 5 to 8 feet long, 2 to 4 feet wide, and 2 to 4 feet high, though the exact size may be smaller or larger and therefore is not a determinative feature of the invention. The connectors may be rigid strips having channels for receiving the sides/floor/top portions. In the alternative, the connectors may be hinge-type mechanisms. The top of the enclosure is open to receive sunlight, or may receive a covering over at least a portion thereof, and one end of the enclosure is constructed from clear or substantially clear material that transmits sunlight to the interior of the enclosure. The rigid side and floor portions are constructed from a dark colored material, such as foam board or plastic or polymer material, such material being capable of absorbing and retaining heat from the sunlight entering the enclosure. This material is not, however, reflective in nature so as to avoid burning of the user's skin.

### BRIEF DESCRIPTION OF THE DRAWINGS

FIG. 1 provides a plan view of a cut-away of the solar tanning system disclosed herein.

FIGS. 2A and 2B provide a cross-sectional view of connectors used with the solar tanning system disclosed herein.

### DETAILED DESCRIPTION OF THE PREFERRED EMBODIMENTS

The invention relates generally to a system for use by individuals to increase their exposure to sunlight during colder weather. More specifically, the invention provides a solar tanning system including a free standing multi-lateral structure, having walls at least 2 feet in height and defining an interior chamber, wherein at least a portion of the walls is sunlight transmissive and the remaining wall portion comprises a dark, non-reflective material, and the chamber has an interior temperature that is higher than the outside temperature. The walls may be rigid, sturdy to withstand wind when used outside. The structure permits sunlight to enter, taking advantage of the same to heat the interior of the enclosure while providing a barrier against cold outside temperatures and wind.

The solar tanning system disclosed herein finds application with respect to the treatment of SAD, Seasonal Affective Disorder, which is a type of diagnosed depression. The disorder stems from a lack of sunlight, which affects the serotonin levels in the body. Seratonin is a factor in and affects the mood of humans. Studies have shown that people between the ages of 15-55 who have SAD may be treated by exposure to sunlight for at least about 30 minutes per day, generally during the dawn or morning hours. Some treatments use artificial light. Others require exposure to direct sunlight. This condition affects people predominantly during the winter months in those areas of the world where cooler or cold weather makes it difficult for people to be outside and experience exposure to direct sunlight for a period of time sufficient to dispel the onset or continuance of the disorder.

In addition to the medical reason to access direct sunlight, many seek to tan prior to vacationing or just to maintain a healthy glow during the winter months when outdoor activity is prevented by the weather. While tanning beds and booths provide one option, natural sunlight remains a preference for many. Additionally, the solar tanning system provided herein may be used to improve the mood and disposition of not only those between the ages of 15-55, that are most affected by SAD, but also younger and older individuals who may be confined to inside environments due to medical reasons, such as by wheelchair, or for other reasons such as exposure to cold or other environmental conditions. The structure provided herein may be sized to accommodate sitting, reclining, standing, or use by multiple people simultaneously.

In one embodiment, the solar tanning system provides a means for an individual to sit or recline outside and be exposed to natural sunlight while not experiencing any of the common drawbacks presented by cooler or cold weather. More particularly, the solar tanning system provides a private, enclosed area, protected by wall, and optionally bottom and top, portions that deflect wind and cooler air but that absorb and retain the warm heat provided by the suns natural radiation. For example, in a colder climate, one may construct the solar tanning system outside, and comfortably sit or lie in the warm interior thereof, without the encumbrance of winter clothing, and get the full benefit of natural sunlight, as prescribed to combat SAD. In addition, the enclosure provides privacy because the walls are constructed from dark material that prevents one outside the box from viewing the interior thereof, i.e., from viewing a person using the box.

It is within the purview of the invention contemplated herein to be constructed without the floor portions, which would still allow the structure to achieve the purpose of blocking cooler or colder weather conditions from affecting the user, as well as provide privacy for the user. Preferably, however, the solar tanning system, in at least one embodiment, includes the bottom portion to enhance the comfort of the user with regard to not having to recline or sit on an exterior surface that may itself be cold or dirty, but more importantly the bottom potion provides a heat-absorbent surface in addition to the wall portions that enhances the overall affect of the solar tanning system with regard to the retention of heat within the system.

Any or all of the wall, floor, top or end portions of the structure may be constructed from fabric or other flexible material, though it is preferred to construct the structure from a rigid, sturdy material to prevent collapse due to wind or other exterior conditions. The material should also be light enough for the user to move and transport the structure, in the constructed state or in the unconstructed state. For example, the structure may be set up by connecting the various wall, floor, top, and/or end portions using appropriate connectors, or by folding and unfolding semi-permanently connected portions. For example, the structure walls, end, top, and floor may be constructed from at least one of foam board, corrugated cardboard, gator board, core board, polymeric material, plastic, or other rigid non-transparent material.

In addition to the foregoing, the solar tanning system is constructed from non-reflective material. The inventor has determined that even in cooler or cold weather, the suns rays, if reflected, are strong enough to burn the skin. Therefore, the solar tanning system includes dark, non-reflective material that absorbs both heat and light energy from the sun's rays and, while reflecting the heat energy, does not reflect the light energy from potentially harmful rays, making it safe to use without the possibility of burning the skin by exposure to reflected light.

In one embodiment, the solar tanning system is used for tanning in natural sunlight during months or periods of time when the temperature is below 65° F. For example, in the northeastern United States, between the months of February and May, the outdoor temperature may range from about 30° F. to about 65° F., though it may dip lower or be higher. Even in these temperatures, an individual can comfortably lay in the solar tanning system and tan, protected from cooler air and wind, and in total privacy. In addition, due to the non-reflective material used to construct the floor or bottom and wall portions, the user may tan safely, assuming other conventional tanning precautions are also used, i.e., lotions, etc.

In another embodiment, the solar tanning system includes an enclosure having one end thereof constructed from a clear or substantially clear material so that the system can be used even early in the day, when the sun is not yet in the full overhead position. In this regard, the user would place the clear or substantially clear end portion of the enclosure toward that direction from which the sun is rising in the earlier hours of the day, generally toward the east, or even setting at the end of the day, generally toward the west. The sun's rays will be transmitted through the clear or substantially clear end portion, still warming the interior of the enclosure, yet preventing wind and cold air from entering the enclosure and causing discomfort for the user.

In addition, the solar tanning system may optionally include a cover. The cover may be made from the same clear or light transmitting material used to construct the end portions, i.e. the cover will allow sun light and heat to be transmitted to the interior of the box. For example, the cover and ends may be constructed for one or more of Plexiglass™ or another clear or substantially translucent plastic or other material, preferably rigid in nature though this is not required. The cover further functions to shield the interior of the box not only from weather but also from the ingress of foreign objects, such as leaves, etc. The cover may extend the full length and width of the box, or it may only partially cover the box. To this end, the cover may be a single piece or may comprise several pieces that may be used individually or in combination to provide the extent of coverage desired by the user. It is generally contemplated that the cover will be fixedly engagable with the end and/or wall portions in any manner provided herein with regard to the connection of wall, end, and floor portions of the box to one another. For example, in one embodiment, the cover may slide into a channel provided in a connector, as shown in the Figures hereof, such that the user may slide the cover forward or backward in accord with achieving the desired comfort. Further, the cover may slide into channels in the end portions, i.e. in a direction from one side wall portion toward the remaining side wall portion, or the cover may slide into channels in the wall portions, i.e. in a direction from one end portion toward the remaining end portion.

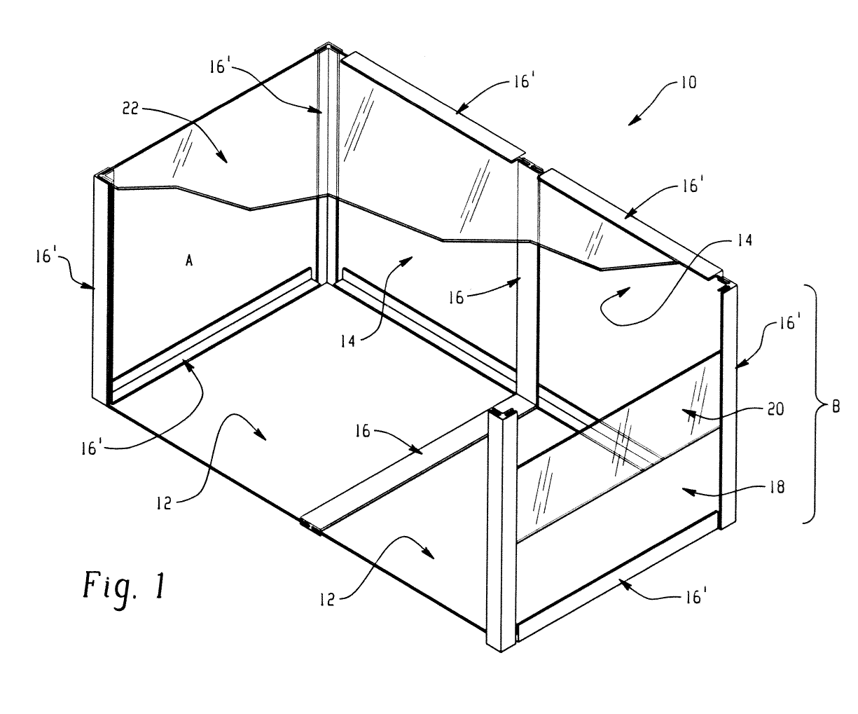

Now then with respect to FIG. 1, there is provided a plan view of a cut-away of a solar tanning system in accord with at least one embodiment of the invention. Shown is the interior of solar tanning system 10 including bottom or floor portions 12 and side wall portions 14. Connecting rods 16, 16′ connect side wall portions 14 to each other and to bottom portions 12. FIG. 1 further shows first end A being constructed from the same material as bottom and side walls portions, 12, 14 respectively. A second end, B, includes a lower section 18 connected to a bottom portion 12 and side wall portions 14, and constructed partially from the rigid, dark colored, non-reflective material used to construct the wall portions. This lower section 18 may be up to about 12 inches high. In this embodiment, abutting lower section 18 is upper section 20, which connects to side wall portions 14 and is constructed from a clear or substantially clear rigid material, such as for example Plexiglass™ or clear or transmissive plastic or any other material that transmits light and is rigid in nature. This upper section 20 may be one piece having a height consistent with the height of a side portion 14 minus the height of lower section 18. In the alternative, the upper section may comprise more than one piece of material, the total height of these pieces being equal to the height of a side portion 14 minus the height of lower section 18. Of course, end section B may be one piece of material that is entirely clear or substantially clear, without a lower section 18 constructed from the rigid, dark, non-reflective material. Also, though in this embodiment end portion A is not light-transmissive like end portion B, it may be constructed from such a material also.

Also shown in FIG. 1 is optional cover 22, shown in cutaway. While cover 22 is shown to be a single piece, it may comprise multiple pieces, of equal or varying width. Further, though cover 22 is shown to engage side wall portions 12, 14, it may engage one or more of side wall portions 14, and/or end portions A and/or B.

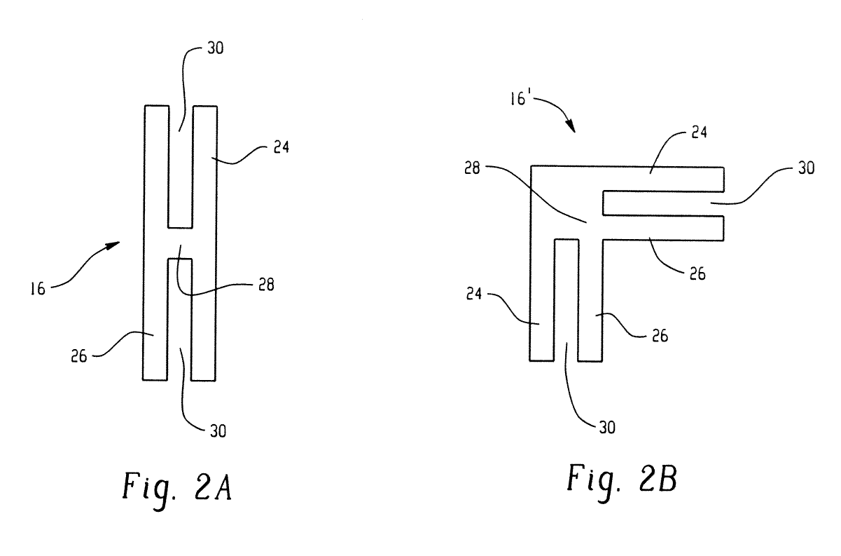

FIGS. 2A and 2B provide examples of one type of connector that may be used to fixedly and removably connect adjoining or abutting edges of side wall, end, bottom, and top portions. Shown in FIG. 2A is a rigid connector 16 defined by parallel, longitudinal sides 24, 26, having a centrally located separator 28. Between sides 24, 26 and on opposite sides of separator 28 are channels 30 that run parallel to sides 24, 26 on the longitudinal axis of the connector. In use, the adjoining or abutting edges may slide into the channels from either end of the connector such that a continuous surface is created by the adjoined wall, end, bottom or top portions. This connector configuration may be used where adjoining wall, end, top or bottom portions extend or lie in the same lateral plane.

FIG. 2B shows a connector 16′, also configured with opposing sides 24, 26 and a center separator 28, though in this configuration the side walls are situated in perpendicular relation to one another and separator 28 forms a corner. Sides 24, 26 again create channels 30. This configuration of connector may be used at the juncture of end-to-side wall, side wall-to-floor, side wall-to-cover, end-to-cover, end-to-floor, i.e. any perpendicular adjoining of box portions.

Alternatively, any other type of connector may be employed. Such as, for example, hinge-type connectors, including Velcro™ straps, hook-and-eye, snap, rigid, or any other type of connector that functions to join contiguous side/end/top/bottom portions. These connectors may be used in that instance where the solar tanning system is intended to be folded before and after use, storage, or transport. For example, using this type of connector, the box may be folded to a size no larger than its largest side/end/top/floor portion. In addition, regardless of the type of connector used, one or more handles may be attached to a surface of the solar tanning system or a carrying case may be provided for ease in transporting or storing the system.

The quadrilateral structure or enclosure may in some embodiments be rectangular, and be suitably sized to comfortably accommodate an adult or teenage human being in the reclining position. For example, the structure, when in the connected state, may be rectangular and have a length of from about 5 to about 8 feet, a width of from about 2 to about 4 feet, and a height of from about 2 to about 4 feet, i.e. a length of at least about 6 feet, a width of at least about 2.5 feet and a height of at least about 2.5 feet, for example a length of about 76 inches, a width of about 32 inches and a height of about 32 inches. Alternatively, the enclosure may be suitably constructed so that an individual may sit therein, have a rectangular or square configuration, or have any other configuration, such as round, hexagonal, octagonal, etc. Of course, the actual dimensions are not limiting, as the enclosure may be constructed to accommodate more than one person, or may be constructed to accommodate one or more persons in the sitting position rather than the reclining position, or any position between sitting upright and lying flat. Similarly, the height of the enclosure may vary, but it is generally high enough to inhibit the ingress of cool or, colder air, such that the interior temperature of the enclosure is warmer than the exterior environment, and to protect the user from being viewed from the exterior of the structure. Therefore, while in one embodiment the enclosure may measure about 5′-8′×2′-4′×2′-4′, these dimensions are not intended to be limitative of the invention as a whole.

The invention has been described with reference to the preferred embodiments. Modifications and variations will occur to others upon reading and understanding the preceding detailed description. It is intended that the invention be construed as including all such modifications and variations that fall within the scope of the appended claims

What is claimed is:

1. A solar tanning system comprising a free standing multi-lateral structure, having walls at least 2 feet in height and defining an interior chamber, wherein a first wall portion is sunlight transmissive and the remaining wall portion comprises a dark, non-reflective material and wherein the chamber has an interior temperature that is higher than an outside temperature.

2. The solar tanning system of claim 1 wherein, the system further includes a floor portion.

3. The solar tanning system of claim 2 wherein, the structure is quadrilateral and the walls include 2 side walls and 2 end walls forming a rectangular chamber, one of the end walls defines the sunlight transmissive portion and the remaining walls are constructed from rigid, dark, non-reflective material.

4. The solar tanning system of claim 1 wherein, the sunlight transmissive portion is constructed from Plexiglass™ or plastic.

5. The solar tanning system of claim 1 wherein, the dark non-reflective material is at least one of foam board, corrugated cardboard, gator board, core board, polymeric material, plastic, or other rigid non-transparent, non-reflective material.

6. The solar tanning system of claim 1 wherein, the structure comprises a foldable structure that in the folded state has dimensions no larger than that of the largest wall portion.

7. The solar tanning system of claim 3 wherein, the floor portions, wall portions and at least one of the at least two end wall portions all have the same perimeter dimension.

8. The solar tanning system of claim 3 wherein, the quadrilateral structure is 5′ to 8′ long, 2′ to 4′ wide and 2′ to 4′ high.

9. The solar tanning system of claim 1 wherein, the structure is 76″ long, 32′ wide and 32″ high.

10. The solar tanning system of claim 1 wherein, the system further includes a cover portion.

11. The solar tanning system of claim 10 wherein, the cover is sunlight transmissive.

12. The solar tanning system of claim 1 wherein, the structure is sized for a person to recline in the interior of the chamber.

13. The solar tanning system of claim 1 wherein, the structure is sized for a person to sit in the interior of the chamber.

14. The solar tanning system of claim 1 wherein, the structure is sized for more than one person to recline or sit in the interior of the chamber.

15. A method of treating a person having Seasonal Affective Disorder (SAD), the method comprising:
providing a free standing multi-lateral structure, having walls at least 2 feet in height and defining an interior chamber, wherein at least a portion of the walls is sunlight transmissive and the remaining wall portion comprises a dark, non-reflective material;
placing the person having SAD in the chamber; and
exposing the person having SAD to natural sunlight that is transmitted through the sunlight transmissive portion of the walls of the structure,
wherein as the sunlight is transmitted into the chamber an interior temperature of the chamber is higher than a temperature outside of the chamber.

16. The method of claim 15 wherein, the structure further includes a floor portion that reflects heat energy from the sunlight in the chamber.

17. The method of claim 15 wherein, the structure further includes a cover that deflects wind and debris from entering the chamber during use.
